(19) United States
(12) Patent Application Publication
(10) Pub. No.: US 2016/0367987 A1
(43) Pub. Date: Dec. 22, 2016

(54) Integrated Multi-modal Imaging and Sensing Techniques to Enable Portable, Label-free, High-specificity, and Scalable Biosensors

(71) Applicant: The Board of Trustees of the Leland Stanford Junior University, Palo Alto, CA (US)

(72) Inventors: Mohammad Amin Arbabian, San Francisco, CA (US); Seyed Miaad Seyed Aliroteh, Milpitas, CA (US); Hao Nan, Stanford, CA (US); Jayant Charthad, Stanford, CA (US)

(21) Appl. No.: 15/165,811

(22) Filed: May 26, 2016

Related U.S. Application Data
(63) Continuation-in-part of application No. 14/939,105, filed on Nov. 12, 2015, now abandoned.
(60) Provisional application No. 62/247,101, filed on Oct. 27, 2015.
(60) Provisional application No. 62/078,899, filed on Nov. 12, 2014.

(51) Int. Cl. B01L 3/00; G01N 33/487

(52) U.S. Cl. CPC B01L 3/502715; G01N 33/48792; G01N 33/48707; B01L 2300/12; B01L 2300/0627; B01L 2200/143; B01L 2200/10; B01L 2200/12

(57) ABSTRACT

An analyte monitoring device is provided that includes a microfluidic channel, where the microfluidic channel is configured for holding a sample under test, an electromagnetic excitation source disposed on a first side of the microfluidic channel, an ultrasonic transducer disposed on a second side of the microfluidic channel, or disposed on the first side of the microfluidic channel, and an appropriately programmed computer, where the electromagnetic excitation source is disposed to induce an thermoacoustic response in the microfluidic channel when holding the sample under test, where the ultrasonic transducer is disposed to receive the thermoacoustic response, where the ultrasonic transducer outputs a voltage to the appropriately programmed computer, where the appropriately programmed computer outputs an analyte value according to the thermoacoustic response.

### CROSS-REFERENCE TO RELATED APPLICATIONS

This application is a continuation-in-part of U.S. patent application Ser. No. 14/939105 filed Nov. 12, 2015, which is incorporated herein by reference. U.S. patent application Ser. No. 14/939105 filed Nov. 20, 2015 claims priority from U.S. Provisional Patent Application 62/247101 filed Oct. 27, 2015, which is incorporated herein by reference. U.S. patent application Ser. No. 14/939105 filed Nov. 20, 2015 claims priority from U.S. Provisional Patent Application No. 62/078899 filed Nov. 12, 2014, which is incorporated herein by reference.

### FIELD OF THE INVENTION

The invention relates to health monitoring. More specifically, the invention relates to a wearable microfluidic analyte monitoring device capable of persistent health monitoring.

### BACKGROUND OF THE INVENTION

Tremendous growth in healthcare costs worldwide has necessitated the need for innovation in the prevention, diagnosis, and treatment of chronic diseases. Digital health monitoring can play a key role in reducing the overall healthcare costs, especially in the case of diseases such as diabetes. Monitoring of advanced health metrics, like blood glucose, face multiple significant challenges. Achieving very high specificity is the most critical challenge for such a biosensor, in addition to high sensitivity, rapid detection, portability, and low cost.

### SUMMARY OF THE INVENTION

To address the needs in the art, an analyte monitoring device is provided that includes a microfluidic channel, where the microfluidic channel is configured for holding a sample under test, an electromagnetic excitation source disposed on a first side of the microfluidic channel, an ultrasonic transducer disposed on a second side of the microfluidic channel, or disposed on the first side of the microfluidic channel, and an appropriately programmed computer, where the electromagnetic excitation source is disposed to induce an thermoacoustic response in the microfluidic channel when holding the sample under test, where the ultrasonic transducer is disposed to receive the thermoacoustic response, where the ultrasonic transducer outputs a voltage to the appropriately programmed computer, where the appropriately programmed computer outputs an analyte value according to the thermoacoustic response.

In one aspect of the invention, the microfluidic channel includes materials such as cross-linked polystyrene, polystyrene (HIPS), polyethylene (LDPE or HDPE), polypropylene, Polytetrafluoroethylene (PTFE), Polyethylene Terephthalate (PET), glass, silicon dioxide, silicon nitride, aluminum oxide, or aluminum nitride.

According to another aspect of the invention, the microfluidic channel includes a living blood vessel or a living plant vessel.

In a further aspect of the invention, the electromagnetic excitation includes an applicator having material such as duraluminum, tin, aluminum, titanium, copper, gold, and platinum. In one aspect the electromagnetic excitation applicator includes an insulating dielectric covering, where the insulating dielectric covering includes a material such as glass, silicon oxide, silicon nitride, aluminum oxide, aluminum or nitride.

According to another aspect, the invention further includes a housing, where the housing includes insulating dielectric materials such as cross-linked polystyrene, polystyrene (HIPS), polyethylene (LDPE or HDPE), polypropylene, Polytetrafluoroethylene (PTFE), or Polyethylene Terephthalate (PET)).

In one aspect of the invention, the electromagnetic excitation source includes an RF source, or an optical source.

In yet another aspect of the invention, a magnetic source is disposed proximal to the sample under test, where the electromagnetic excitation source is disposed to induce a magnetoacoustic response in the microfluidic channel when holding the sample under test, where the ultrasonic transducer is disposed to receive the magnetoacoustic response, where the ultrasonic transducer outputs a voltage proportional to the magnetoacoustic response. In one aspect, the appropriately programmed computer is configured to match a waveform shape and amplitude of the thermoacoustic or the magnetoacoustic response in a time domain to known signatures from analytes of interest. In another aspect, the appropriately programmed computer is configured to match a frequency shape and phase content of the thermoacoustic or the magnetoacoustic response in a frequency domain to known signatures from analytes of interest. In a further aspect, the sample under test includes a contrast agent, where the contrast agent interacts with an analyte that is disposed to provide an identifiable thermoacoustic response or a magnetoacoustic response, where the analyte includes a biomolecule, cells, or synthetic compounds. Here, the contrast agent includes materials such as iron oxide, super-paramagnetic iron oxide nanoparticles (SPION), where the SPION is selected from the group consisting of hematite (α—Fe₂O₃), maghaemite (γ—Fe₂O₃), or aluminum substitutes in Iron Oxide (ε—AlₓFe₂—xO₃).

According to one aspect of the invention, the electromagnetic excitation source is modulated, where the modulated electromagnetic excitation source is disposed to generate a modulated thermoacoustic response.

In another aspect of the invention, the ultrasonic transducer includes a MEMS sensor.

In a further aspect of the invention, the sample under test includes blood, saliva, urine, bodily fluids, agricultural fluid extracts, water system, sewer, or industrial fluid extracts.

According to another aspect of the invention, the thermoacoustic response includes an acoustic shock wave in the microfluidic channel formed by microthermal heating and expansion.

### BRIEF DESCRIPTION OF THE DRAWINGS

FIG. 1 shows an unobtrusive, non-Invasive, and persistent health monitoring and medication modulating smartwatch and vital signs, physical activity, and environmental factors, where the device is capable of measuring advanced and clinically relevant health metrics, including biomarkers concentrations, to make an informed decision for automatically tuning and modulating your medications as necessary, according to one embodiment of the invention.

FIG. 2 shows pills that employ techniques in nanotechnology to deliver drugs that can later be activated by non-invasive means such as dectromagnetic and/or ultrasonic waves, where contrast agents, for monitoring hard-to-detect biomarkers can be simultaneously delivered. This allows the external device, the smartwatch, to monitor relevant biomarkers in order to decide when it is necessary to activate the drugs, according to one embodiment of the invention.

FIGS. 3A-3B show a control experiment where it is confirmed that very low background Thermoacoustic signals are produced by the microcapillary itself (see FIG. 3A). FIG. 3B shows the successful Thermoacoustic detection of salt water inside a 150 μm microcapillary.

FIG. 4 shows a (blood) sample flowing through a microfluidic channel is irradiated by microwave applicators, where analytes and biomarkers, potentially labeled with contrast agents, absorb the microwave and undergo thermoelastic expansion producing acoustic waves that are captured by MEMS ultrasound sensors. The sensors have sufficient acoustic bandwidth to obtain the necessary resolution for the distinction of multi-analytes in order to avoid ambiguity during analysis, according to one embodiment of the invention.

FIG. 5 shows the parametrized, Thermoacoustic response of a material after pulsed microwave excitation, according to one embodiment of the invention.

FIGS. 6A.-6C show the Thermoacoustic response of various materials, including glycerin and isopropyl alcohol, was tested 10 times and the experiment repeated on another day as a measure of variability. The results were identical for the same material but varied between materials with regards to amplitude and waveform shape, according to one embodiment of the invention.

FIGS. 7A-7C show boxplots (7A, 7B) with statistically significant differences in the Thermoacoustic response of glycerin, isopropyl alcohol, and salt water both in terms of amplitude and waveform shape. Thus it is fundamentally possible to distinguish different materials using the Thermoacoustic technique. Day to day variations (e.g. “glycerin1” and “glycerin2”) were small and the measurements were consistent. The milk and sugar water samples exhibited lower signal-to-noise (SNR) ratios that resulted in greater variance of their responses. By employing more sensitive (MENIS) ultrasound sensors the SNR and variance of these samples can be improved, according to the current invention.

FIGS. 8A-8D show the processing techniques for manufacturing Thermoacoustic microfluidic chips, according to embodiments of the invention.

FIGS. 9A-9E show the processing techniques for manufacturing Thermoacoustic microfluidic chips, according to embodiments of the invention.

FIGS. 10A-10E show the processing techniques for manufacturing Thermoacoustic microfluidic chips, according to embodiments of the invention.

FIGS. 11A-11F show the processing techniques for manufacturing Thermoacoustic microfluidic chips, according to embodiments of the invention.

FIG. 12A-12F show the processing techniques for manufacturing Thermoacoustic microfluidic chips, according to embodiments of the invention.

FIG. 13A-13C show further embodiments of the current invention.

FIG. 14A-14B show further embodiments of the current invention.

FIGS. 15A-15C show further embodiments of the current invention.

### DETAILED DESCRIPTION

The current invention provides a multi-modality sensing device that uses the electromagnetic and thermo-elastic (Thermoacoustic) response of target compounds for achieving a high specificity. According to one embodiment, the invention is scaling down to smaller dimensions using higher acoustic frequencies, where a next-generation biosensor for the non-invasive and continuous monitoring of advanced health metrics like blood glucose, medication concentrations and eventually circulating tumor cells is provided. Demonstrated herein are two critical aspects of a Thermoacoustic biosensor. The first is reduction, by 4 orders of magnitude, in the required operating peak power, which allows 10 kV vacuum sources to be replaced by 48V solid-sate devices consuming 0.5 W average power. The second is experimental confirmation that both acoustic and electrical properties of samples or analytes contribute to their unique identification through Thermoacoustic sensing.

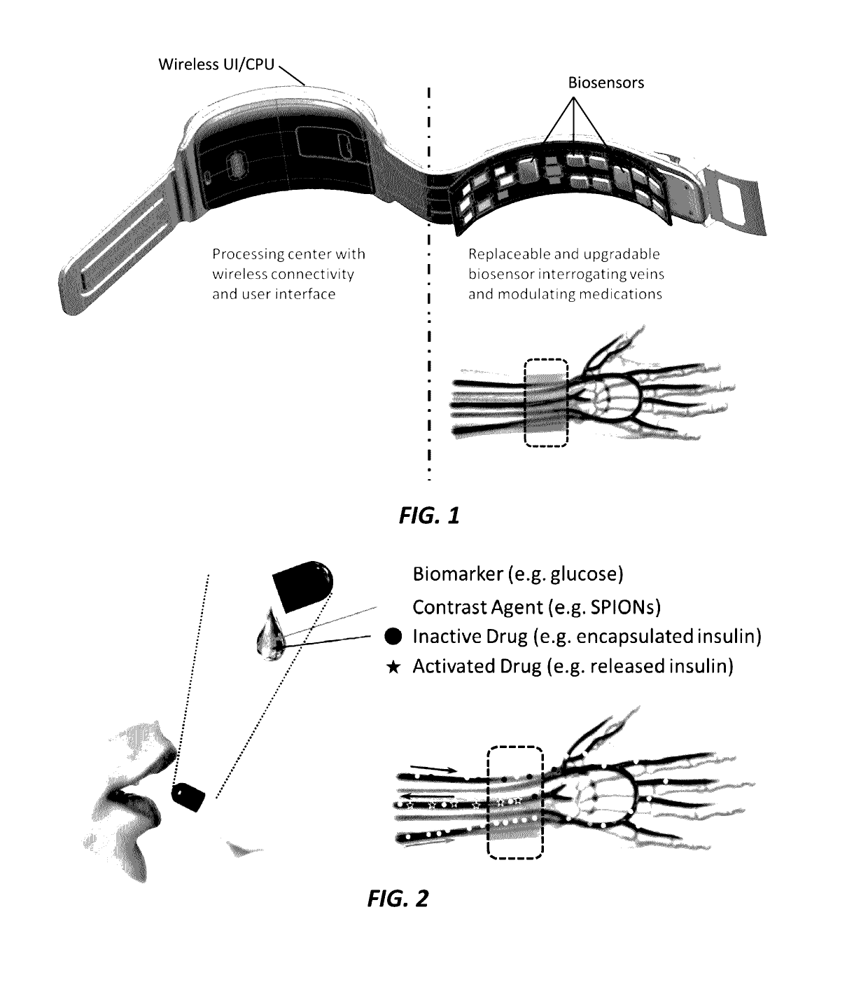

The current invention enables proactive, routine, and unobtrusive health monitoring that facilitates early diagnosis and preventative maintenance, thus reducing doctor visits and hospital waiting lines. The invention includes wearable electronics, internet-of-things connectivity, data on the Cloud, and emerging biosensing technologies configured in an affordable smartwatch, as shown in FIG. 1, with the required awareness and capability to dynamically control activity of required medications by non-invasively and persistently monitoring:
Common vital signs
   Pulse (e.g. photoplethysmogram)
   Heart Activity (e.g. Electrocardiogram)
   Blood pressure
   Body Temperature
   Skin Conductance
Your activity and environment
   Motion, walking, running
   Sweat
   Altitude
Clinically relevant, advanced health metrics including biomolecule and biomarker concentrations
   Blood glucose level
   Concertation of medications such as antibiotics
   Presence of any circulating tumor cells

Currently, smartwatches can monitor some common vital signs and physical activity. In general, low power and portable sensors for monitoring pulse, electrocardiogram, blood pressure, and temperature, motion, sweat and skin conductance are commonplace. The current invention provides advanced biosensing capabilities that enable the synchronous monitoring of clinically relevant biomarkers along with vital signs and activity that shifts the paradigm from treatment and therapy to continual monitoring and preventive care. The current invention enables a move away from the “one-size fits all” mentality of current pharmaceuticals to the personalized and dynamically tuned pharmaceuticals of the future.

Early diagnosis of cancer is one example where biomarkers such as circulating-tumor-cells (metastatic cancer), Prostate-Specific Antigen (prostate cancer), and others need to be monitored continuously. Early warnings for heart disease and stroke may also be detected by long term monitoring of biomarkers such as B-type Natriuretic Protein (BNP), Cardiac Troponin (cTn), and Ischemia-Modified Albumin (IMA). More generally, new applications will emerge once they are enabled by new biosensing technologies.

Because the Thermoacoustic technique of the current invention relies on electromagnetic excitation, achievable with integrated electronics, and acoustic detection, achievable with MEMS sensors, it is implemented with existing semiconductor technologies to provide biosensors based on Thermoacoustic phenomenon.

The two key defining attributes of a biosensing platform are non-invasive, in-vivo detection (and drug modulation) and Label-free detection. Each of these two major attributes is very challenging to obtain in itself and hence three categories of monitoring styles are embodied by the invention:
Style I—Ex-vivo, label-free detection
Style II—Non-invasive, in-vivo, label-assisted detection
Style III—Non-invasive, in-vivo, label-free detection

The first style emphasizes label-free detection, ex-vivo, in the controlled environment of a microfluidic biosensing chip. This permits various sample filtering mechanisms, including ultrasound techniques, that can remove large quantities of known undesired biomolecules and cells from the sample under test, enabling higher specificity and label-free detection simultaneously. For example, it is common to filter out white and red blood cells from a blood sample on a microfluidic chip and only analyzing the rest of the sample. In the Thermoacoustic biosensor in particular, ultrasound sensors and RF applicators are integrated into the microfluidic channels. This reduces the field of view, enhancing signal-to-noise ratio (SNR) and ultimately enabling greater specificity in a label-free detection scheme. Multiple biomarkers can be monitored with one Thermoacoustic biosensor as long as the each exhibits a unique Thermoacoustic signature, where the combined electromagnetic and mechanical properties of each biomarker are unique.

The second monitoring style emphasizes non-invasive, in-vivo operation without the need for extracting a blood sample. This necessitates a larger separation between ultrasound sensors and RF applicators located on the skin and the biomarkers located in the blood stream. This larger separation means more attenuation of ultrasound at higher acoustic frequency, and less electromagnetic focusing, which would limit resolution and detection capabilities. To compensate for this, safe and biocompatible Thermoacoustic contrast agents are ingested in the form of a pill as in FIG. 2. These contrast agents can be synthesized to target the desired biomarker, for example through antibody markers, and produce large and detectable Thermoacoustic responses under excitation. Multiple biomarkers can be monitored with one Thermoacoustic biosensor as long as the necessary label and antibody can be manufactured for each one.

The third and final monitoring style combines the previous two and simultaneously achieves non-invasive and label-free detection. This requires highly refined and optimized system design which is likely to be only possible in particular applications and for particular biomarkers. Thus each compatible biomarker requires its own uniquely designed and optimized biosensor.

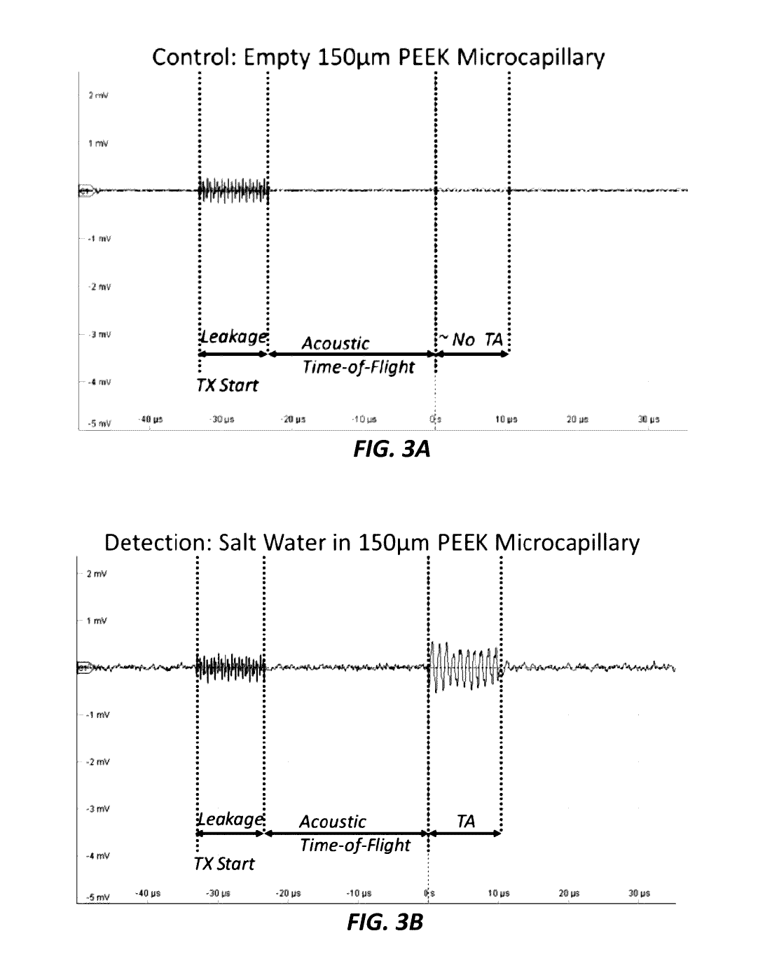

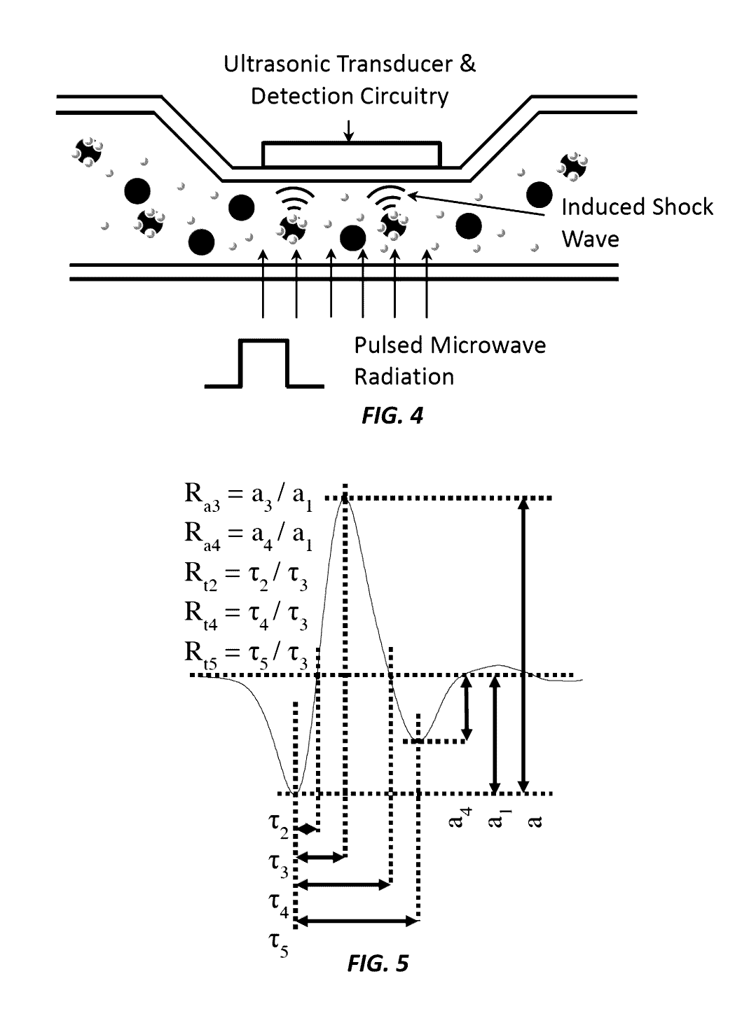

FIGS. 3A-3B show a control experiment where it is confirmed that very low background Thermoacoustic signals are produced by the microcapillary itself (see FIG. 3A), FIG. 3B shows the successful Thermoacoustic detection of salt water inside a 150 μM microcapillary. Thermoacoustic sensing can exploit both mechano-acoustic properties and electromagnetic properties of samples in order to differentiate them. It is well know that electromagnetic properties can be exploited for material detection. For instance, measurement of dielectric properties over a several GHz of bandwidth can be used identify the concentration of sugar or glucose in water. In principle Thermoacoustic sensing, which is also dependent on dieletric properties, can also capture this. However when a complex solution, such as blood, is tested the dielectric measurements cannot reliably estimate glucose concentrations. Here the complexity of the solution has introduced more variability than can be captured by electromagnetic properties alone. However Thermoacoustic sensing, having more degrees of freedom through mechano-acoustic properties, is better equipped to deal with such complex solutions. Moreover, acoustic detection allows for high resolution (10 μm) sensing that can zoom in on the components found within a complex solution. Thus a Thermoacoustic detector can identify multiple analytes, as in FIG. 4, minimizing ambiguity in the analysis. In fact, ultrasound can be used for acoustic microscopy where an entire cell is imaged at high resolution. However ultrasound microscopy lacks the electromagnetic contrast provided by the Thermoacoustic techniques. In biosensing applications, the ultrasound sensor's resolution would lie between a coarse detector and a complete imager, as it needs to exploit just enough resolution for multi-analyte detection.

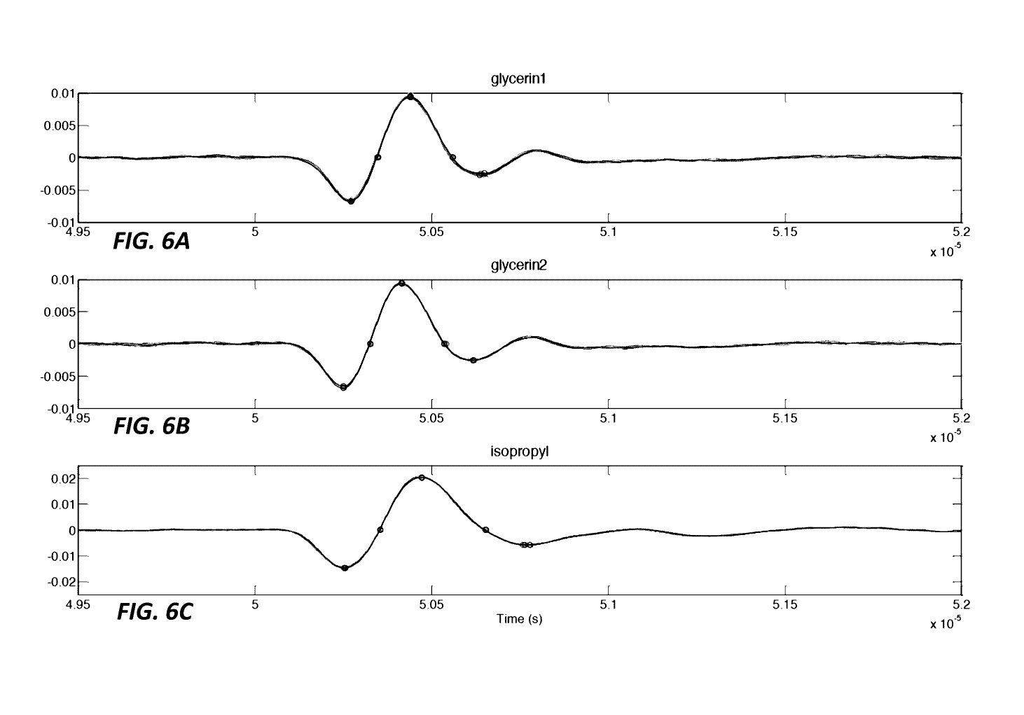

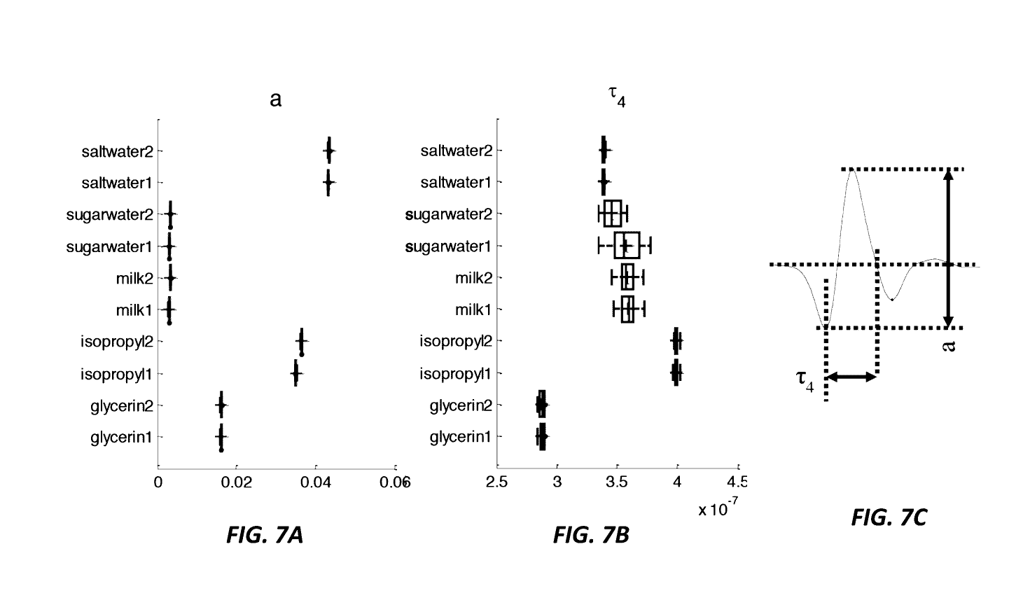

Different materials based on their Thermoacoustic response to pulsed microwave excitation are identified, as illustrated in FIG. 5. By using a single microwave excitation frequency, signals such as those in FIGS. 6A-6C were obtained that reflect differences in acoustic properties only. As illustrated in FIGS. 7A-7C these signals show statistically significant differences in waveform amplitude and shape. Furthermore, the measurements were repeated at a different time with consistent results, showing small variability from day to day. These findings show that mechano-acoustic properties do indeed provide contrast and useful information about an analyte beyond what would already be available through its electromagnetic properties. In summary, Thermoacoustic technique is able to obtain contrast and specificity through both electromagnetic and mechano-acoustic properties as well as achieving sufficient resolution to avoid ambiguities in captured signals arising from multiple analytes. This combination of features could not be obtained by electromagnetic and ultrasonic detection techniques separately.

Contrast agents are commonplace in imaging applications, where they improve image quality through enhanced contrast. In Thermoacoustic imaging and sensing this is achieved through the interaction of the contrast agent with microwave excitation to more effectively convert the electrical energy into heat. Iron oxide nanoparticles are small enough to have a single ferromagnetic domain where their magnetization can flip randomly due to thermal fluctuations. The characteristic time between such flipping events is called the Neel relaxation time. Such particles are called Superparamagnetic Iron Oxide Nanoparticles (SPIONs). Macroscopically, a solution containing such particles exhibits a real and lossless permeability at most frequencies except for a particular resonance frequency, related to the reciprocal of the Neel relaxation time, where the permeability becomes complex and lossy. At this resonant frequency electromagnetic energy is converted into heat. This electrical-to-thermal energy conversion produces significantly larger Thermoacoustic pressure waves than pure water, which constitutes the majority of cells and tissue. It is important to note that SPIONs have been exploited in other imaging applications, including MRI, and there is a growing effort in their commercialization.

Microscopically, SPIONs convert microwave energy into heat via spin resonances. Here, unpaired electrons spin under an applied external magnetic field (e.g. a miniature permanent magnet), creating a magnetic moment similar to nuclear spins in MRI. This phenomenal is called ferromagnetic resonance (FMR). The unpaired electrons align either parallel or anti-parallel to the external magnetic field, with the magnetic moments precessing at the Lamor frequency, which lies within the microwave frequency region:

ω₀=−γμ₀H_{eff} (1)

Where γ is the gyromagnetic ratio, μ₀ is the permeability of free space, and H_{eff} is the effective magnetic field strength. Materials with asymmetries, impurities, and remnant magnetization can exhibit FMR without the application of an external magnetic field. By modifying the size, material composition, and shape of the nanoparticles it is possible to tune the resonant frequency of FMR. This allows the production and use of multiple contrast agents simultaneously, where each is detected at its own unique resonant frequency without disturbing other contrast agents. Thus, such a label-assisted biosensing scheme is able to simultaneously track multiple biomarkers. Moreover, these contrast agents can be paired with the appropriate anti-body in order to target any desired biomolecules or cell. In this way a general purpose, non-invasive, in-vivo, label-assisted biosensing platform with a single, common detection platform, the smartwatch, and multiple customized contrast agents in pill format is enabled.

With Thermoacoustic sensing, the same contras-agent SPIONs designed for electrical-to-thermal energy conversion is exploited for drug modulation. This dual use of SPIONs makes the Thermoacoustic technique more competitive than alternative sensing technologies. The feasibility of biocompatible polymers that undergo large structural changes with modest, local, temperature changes around 37° C. has been demonstrated. These changes include shrinkage, swelling, and transitions in wettability, which have been harnessed to produce drug delivery carriers. In particular, soft-material/hard-material hybrids, where magnetic metals or oxides are embedded in a temperature-responsive polymer matrix are used to link thermal sensitivity with selective electromagnetic activation through resonance. This permits reliable drug release on demand and slow drug modulation applications.

Linear polypeptides made of amino acid monomers also exhibit shape changes at a well-defined collapse temperature determined by the hydrophilicity of the respective amino acid. For example, valine monomers have a collapse temperature of 24° C. while for glycine monomers this increases to 55° C. By combining these amino acids it is possible to engineer a polymer with a desired collapse temperature. For example, polymers having Valine-Proline-Glycine-Valine-Glycine repeats exhibit a collapse temperature of 26° C. which is increased to 42° C. after randomly substituting 50% Valine, 30% Glycine and 20% Alanine for the second Valine in the repeats. A widely used temperature-responsive polymer is poly(ethylene oxide)-poly(propylene oxide)-poly(ethyleneoxide) or PEO-PPO-PEO for short. Another commonly used temperature-responsive polymer is Poly(N-isopropylacrylamide) or PNOPAAm for short. Both of these polymers as well as iron oxide nanoparticles have been shown to be readily and safely digested and gradually cleared from body.

A self-assembly strategy of aqueous nanocapsules intended for drug deliver on demand under magnetic stimulation has been shown. Magnetic heating of these magnetic nanocolloids was shown to gradually increase the temperature of the entire solution by several degrees at a rate of 0.1° C./s to 1° C./s. This would correspond to a local heating rate, in the near vicinity of the nanoparticles, on the order of 100° C./s.

Moreover, evidence from microscopy suggested that the local temperature rise of the nanoparticles is several hundred degrees Celsius even if the solution temperature increased by several degrees only. This evidence shows that very efficient and localized heating, required for activating the temperature-responsive polymers, is possible. In fact, experiments were performed to compare drug release triggered by local magnetic heating against the response to external heating of the entire solution. These results point to the feasibility of selective drug delivering and modulation via electromagnetic triggers. According to one aspect of the invention, modification of the triggering technique from simple magnetic heating to ferromagnetic resonance heating in order to take advantage of multi-frequency operation enables simultaneous and independent control of multiple drugs.

For the detection and differentiation of cells, microparticles, nanoparticles, and potential contrast agents a Thermoacoustic microfluidic platform a required resolution (˜10 um) and SNR is required. Several factors must be considered, including:
The materials from which a microfluidic chip is constructed must not produce Thermoacoustic signals that would otherwise interfere with the desired signals originating from the samples.
The microfluidics channel width and height (˜100 um) are smaller than capabilities of Printed-Circuit-Board technology and larger than standard microfabrication techniques.
Design and construction must be fairly rapid to enable prototyping and iterative design.
The construction should be economical and compatible with our modest resources.

To address these constraints a two-part approach includes a process suitable for prototyping and a process suitable for mass-production. The prototyping process uses lower processing temperatures, and can incorporate higher performance materials such as fused silica (glass), contains manufacturing steps, and permits the peeling, cleaning, and resealing of the microfluidic channels. In contrast, the mass-production process uses higher processing temperatures, incorporates economical and high-volume materials, contains more complex manufacturing steps, and produces permanently sealed microfluidic channels. Observations and experimental test on various materials include:
HDPE has acoustic impedance of 1.4 Mrayls, which matches closely to water, 1.5 Mrayls, and oil, 1.3 Mrayls, whereas most other plastics and Rexolite have larger acoustic impedance mismatches.
It is possible to strongly bond HIPS and Rexolite by sanding Rexolite and melting HIPS (with hot air).
It is possible to bond HDPE and Rexolite by sanding Rexolite and melting HDPE (with hot air).
It is possible bond HDPE and HIPS, as well as HDPE and HDPE, by melting them (with hot air).
Sanding or chemically etching glass allows it to bond to HIPS and HDPE by melting them (with hot air).
It is possible to polish Rexolite to make it transparent like glass, which is extremely useful for inspection of a microfluidic channel under a microscope.
   Can be achieved by sanding, polishing, buffing, and finally heating Rexolite around 180° C. in the absences of oxygen (to avoid oxidation) as confirmed experimentally.

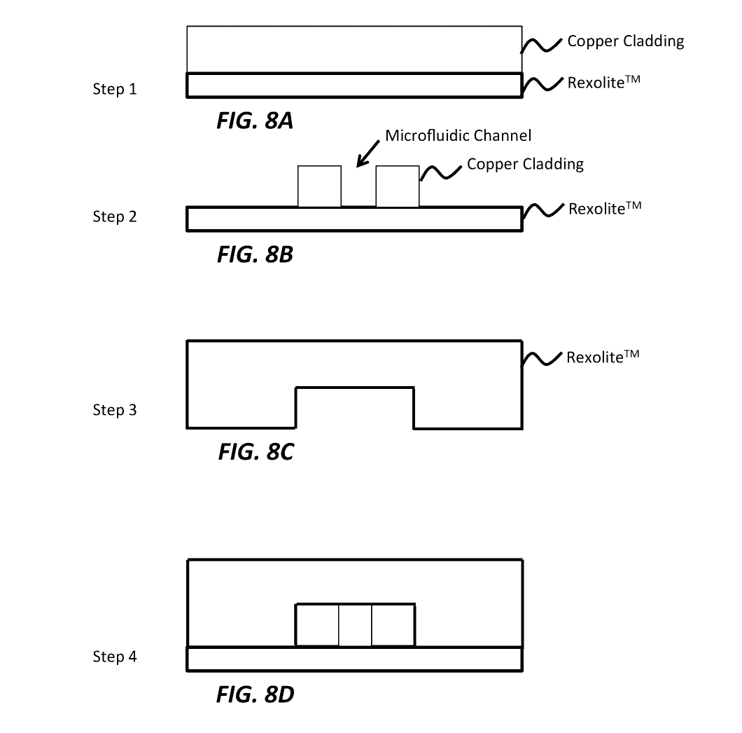

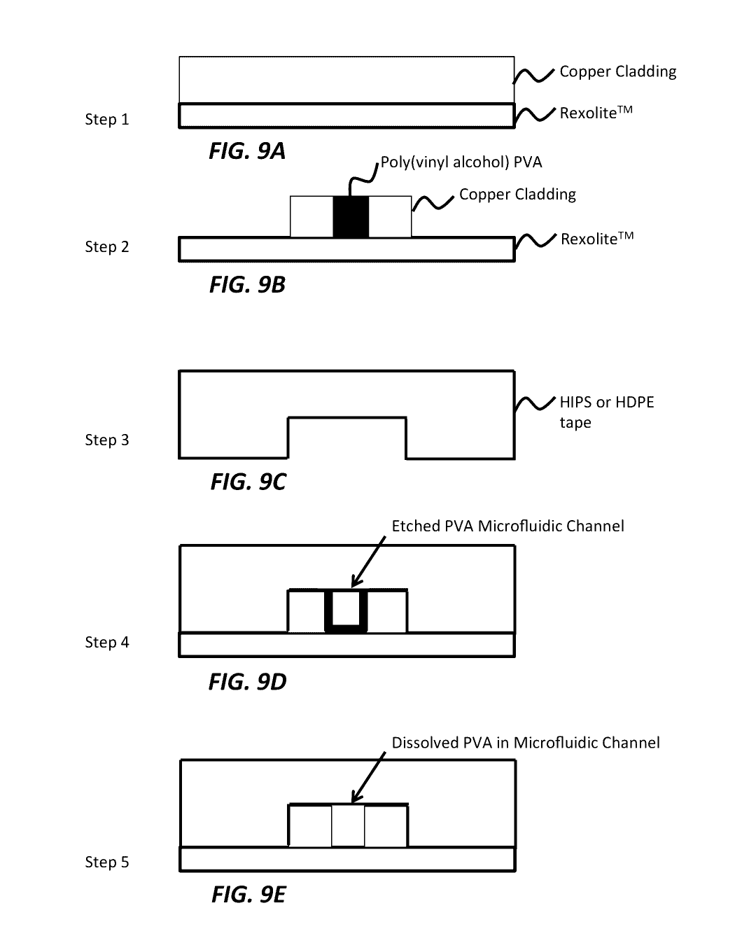

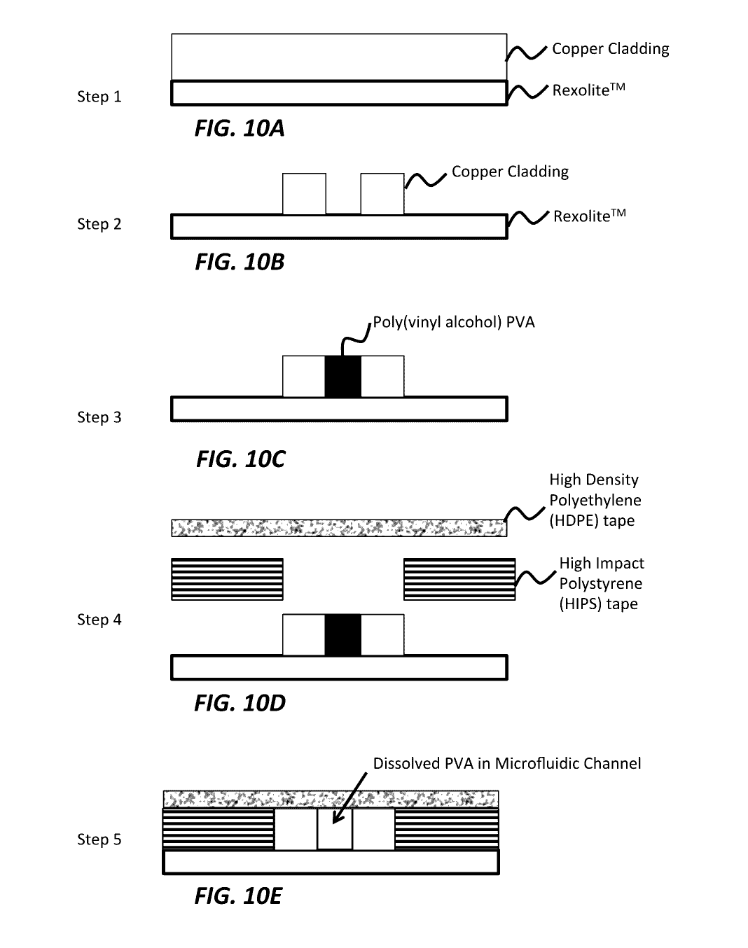

When considering these aspects, three main processing techniques for manufacturing Thermoacoustic microfluidic chips are provided. As shown in FIGS. 8A-10E, these three processes that include Rexolite copper clad boards as the starting substrate. Modifications of the processes shown in FIGS. 9A-9E and FIGS. 10A-10E enable the use of fused silica. In one exemplary embodiment, microscope glass slides pre-coated with metals such as gold, copper, or aluminum are copper electroplated to 10 um-100 um thick metal layers. Finally, further nickel and gold electroplating (thin layers) is performed for passivation and solder compatibility. The rest of the processing steps are the same as in FIGS. 8A-8D. Processes shown in FIGS. 9A-9E and FIGS. 10A-10E, where HIPS is used entirely or as a bonding layer can have their microfluidic channel caps peeled using Limonene. This permits cleaning of the channel or re-machining if required. Afterwards steps 3-5 in these processes can be repeated to reseal the channel.

As shown in FIGS. 8A-8D, the process steps for manufacturing Thermoacoustic microfluidic chips includes:
5. Using a Rexolite copper clad.
6. Etch/mill/micro-machine copper and drill holes. Polish the top of the Rexolite with buffing, acetone, or limonene.
7. Mill or micro-machine another Rexolite piece or injection mold it. Polish to transparency.
8. Attach the two pieces using Rexolite welding adhesive. Epoxy microfluidics ports. Polish top of Rexolite for microscope inspection. Ultrasound is transmitted from the bottom with some attenuation.

As shown in FIGS. 9A-9E, a further embodiment of the process steps for manufacturing Thermoacoustic microfluidic chips is shown that includes:
6. Using a Rexolite copper clad.
7. Etch/mill/micro-machine copper and drill holes. Polish the top of the Rexolite with buffing, acetone, or limonene.
8. Melt and deposit/spin PVA inside the channel. Can use water to erase mistakes or as an etchant to remove PVA using a mask (such as Micron archival ink pen). Planarize by simple sanding if necessary.
9. Heat press HIPS or HDPE tape to seal and form a channel. HIPS transmits ultrasound with some attenuation while HDPE has minimal acoustic loss.
10. Epoxy microfluidics ports and place device in water to dissolve PVA inside the channel. Polish bottom of Rexolite for microscope inspection.

FIG. 10 shows another embodiment of the process steps for manufacturing Thermoacoustic microfluidic chips is shown that includes:
6. Using a Rexolite copper clad.
7. Etch/mill/micro-machine copper and drill holes. Polish the top of the Rexolite with buffing, acetone, or limonene.
8. Melt and deposit/spin PVA inside the channel. Can use water to erase mistakes or as an etchant to remove PVA using a mask (such as Micron archival ink pen). Planarize by simple sanding if necessary.
9. Cut (with laser cutter) HIPS tape and place it outside the channel as a bonding layer. Heat press HDPE tape, sandwiching the HIPS layer for bonding. HDPE is ideal for ultrasound transmission.
10. Epoxy microfluidics ports and place device in water to dissolve PVA inside the channel. Polish bottom of Rexolite for microscope inspection.

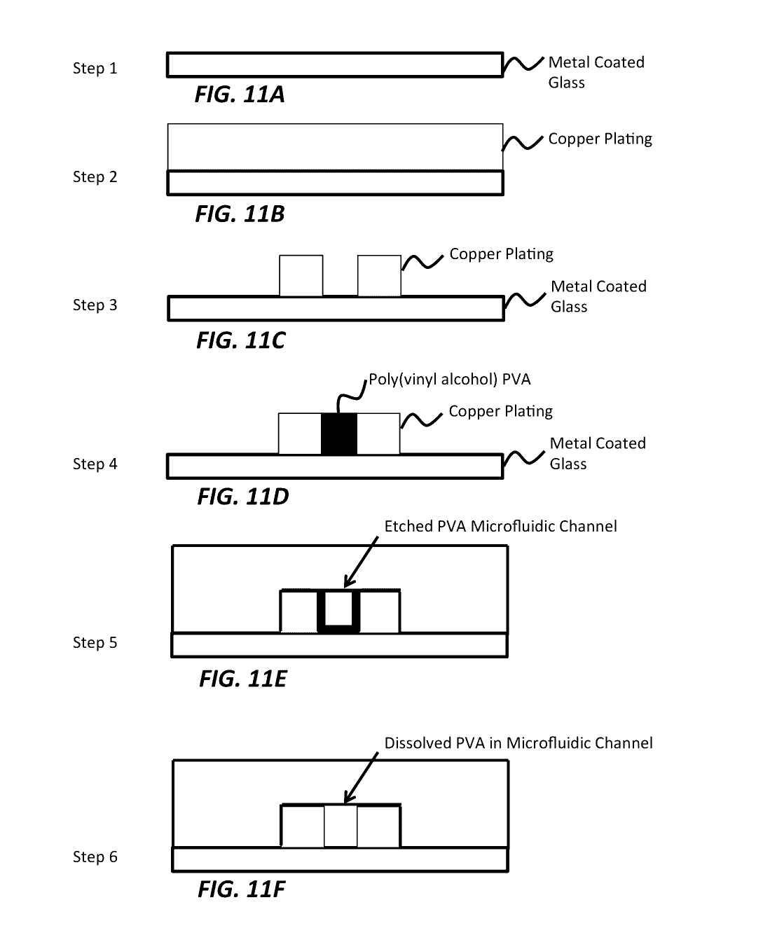

FIGS. 11A-11F show other embodiments of the process steps for manufacturing Thermoacoustic microfluidic chips is shown that includes:
7. Glass is etched or plasma activated. Then an electroplating seed layer (TiW) is sputtered to create metal coated glass. Metal coated glass slides are available for purchase.
8. Electroplate copper to achieve 10 um to 100 um thick metal layers. Electroplate for Ni and then Au for passivation and soldering.
9. Etch/mill/micro-machine copper and drill holes. Polish the top of the glass, inside the channel, by buffing.
10. Melt and deposit/spin PVA inside the channel. Can use water to erase mistakes or as an etchant to remove PVA using a mask. Planarize by simple sanding if necessary.
11. Heat press HDPE tape to seal and form a channel. HDPE is ideal for ultrasound transmission.
12. Epoxy microfluidics ports and place device in water to dissolve PVA inside the channel.

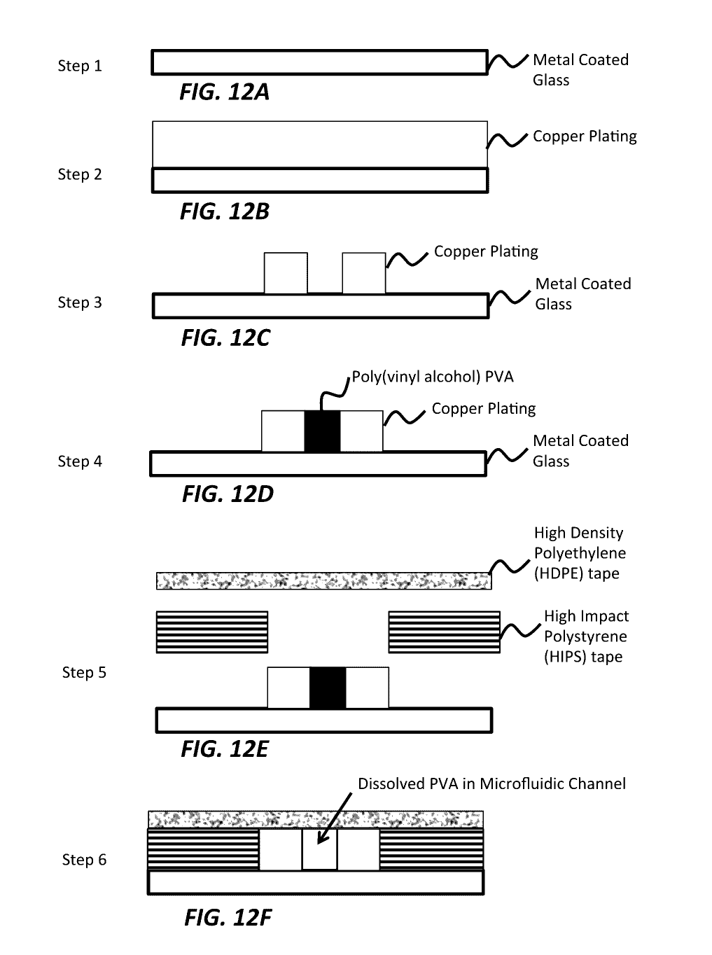

FIGS. 12A-12F show another embodiment of the process steps for manufacturing Thermoacoustic microfluidic chips is shown that includes:
7. Glass is etched or plasma activated. Then an electroplating seed layer (TiW) is sputtered to create metal coated glass. Metal coated glass slides are available for purchase.
8. Electroplate copper to achieve 10 um to 100 um thick metal layers. Electroplate for Ni and then Au for passivation and soldering.
9. Etch/mill/micro-machine copper and drill holes. Polish the top of the glass, inside the channel, by buffing.
10. Melt and deposit/spin PVA inside the channel. Can use water to erase mistakes or as an etchant to remove PVA using a mask. Planarize by simple sanding if necessary.
11. Cut (with vinyl cutter) HIPS tape and place outside channel for bonding layer. Heat press HDPE tape, sandwiching the HIPS layer for bonding. HDPE is ideal for ultrasound transmission.
12. Epoxy microfluidics ports and place device in water to dissolve PVA inside the channel.

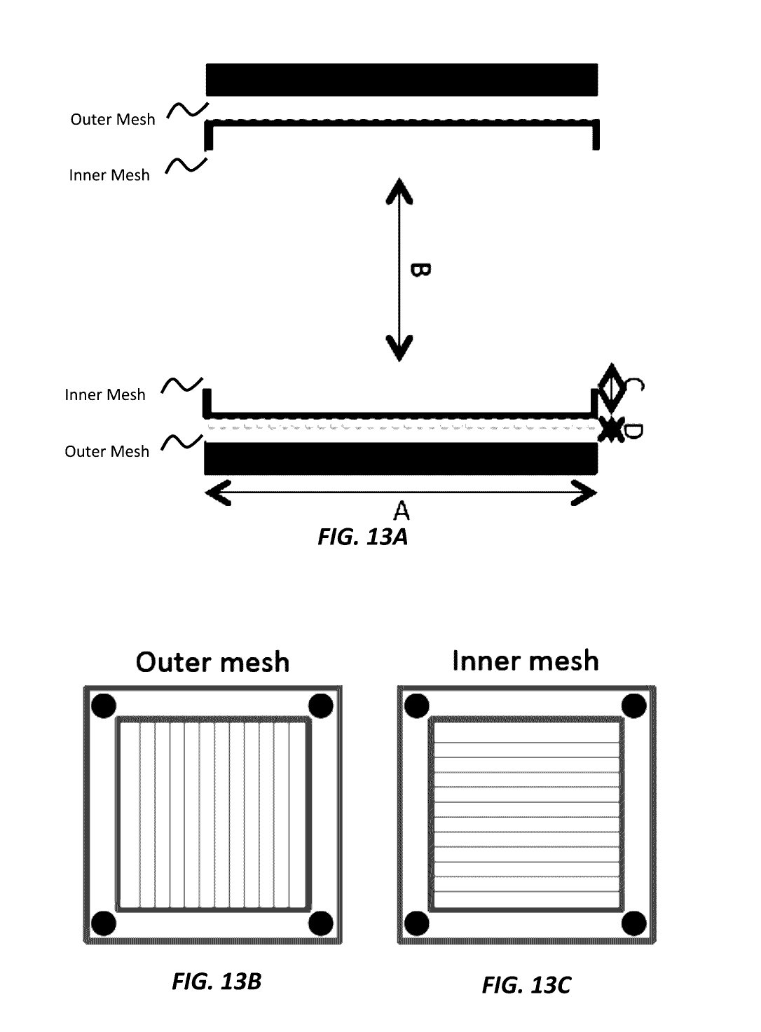

FIGS. 13A-13C show a further embodiment of the current invention having 2-phase, integrated TX and RX subsystems, which includes:
Outer mesh drives differential RF.
Inner mesh shields CMUT and also can be used for electro-wetting.
In another variant the inner mesh is removed and the grounded top-plate of the CMUT replaces its function.
In another variant both meshes are removed
   The top plate passes through an RF choke (parallel LC) before connecting to the RX chain and the bottom substrate passes through an RF choke (parallel LC) before connecting to negative VDC for CMUT bias.
   Both botttom substrate and top plate pass through two RF shorts (series LC) before connecting to RF+ (or RF− for the other unit)
   A=B=10 um to 50 um. C=D=0.1 um
   mesh: 0.1 um×0.1 um copper/gold/metal lines space 1 um apart on a grid.

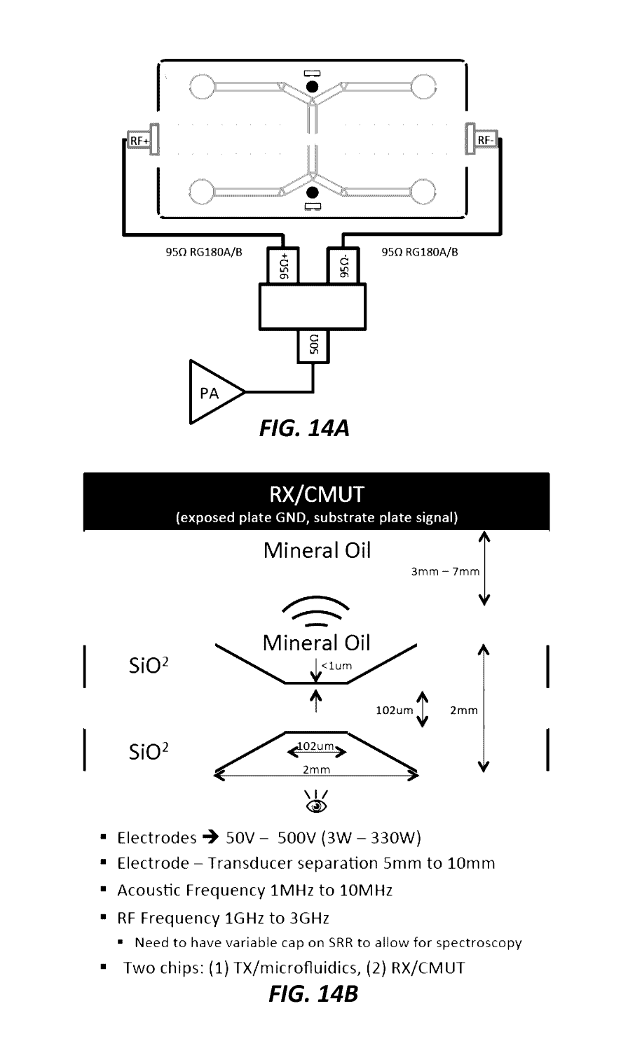

FIGS. 14A-14B show another embodiment of the invention having separate TX and RX subsystems, which include:
TA signal reflects average electrical and mechanical properties of a nL volume sample
Visual inspection with microscope from below
Measure acoustic signals from above
102 um×102 um lines, 108 um spacing→94Ω
   <0.02 dB/cm loss or an expected loss<0.2 dB

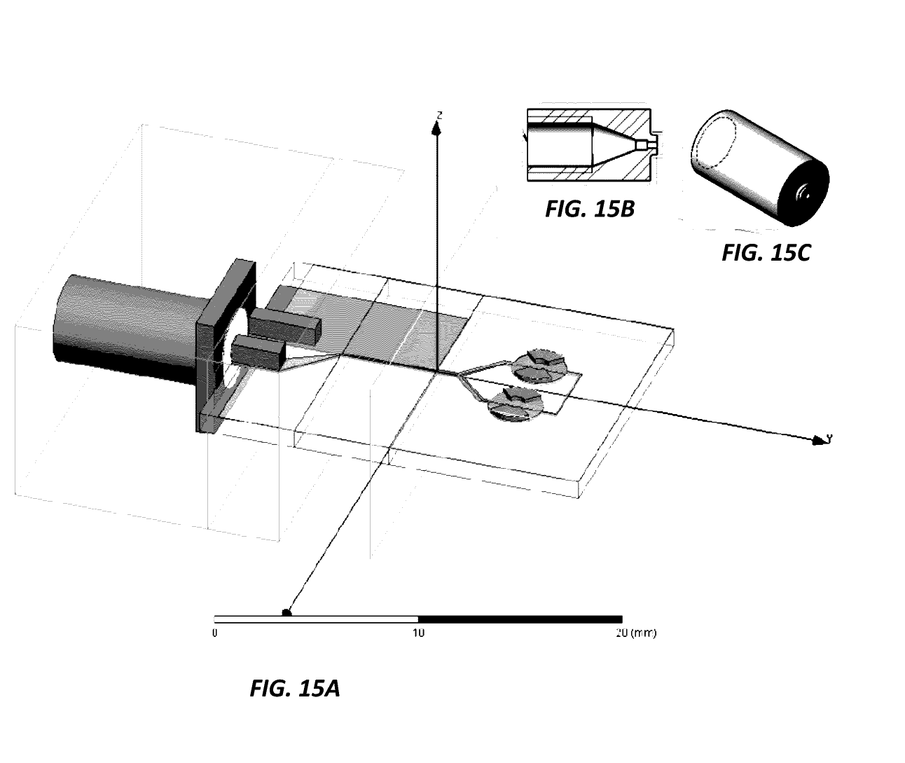

FIGS. 15A-15C show an additional embodiment of the invention having:
Planar design that solders directly to SMA connector.
Also can epoxy two microfluidic ports from LabSmith so that micro-capillaries/syringes can connect to the chip.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations are considered to be within the scope and spirit of the present invention as defined by the following claims and their legal equivalents.

What is claimed:

1) An analyte monitoring device, comprising:
a) a microfluidic channel, wherein said microfluidic channel is configured for holding a sample under test;
b) an electromagnetic excitation source disposed on a first side of said microfluidic channel;
c) an ultrasonic transducer disposed on a second side of said microfluidic channel, or disposed on said first side of said microfluidic channel; and
d) an appropriately programmed computer, wherein said electromagnetic excitation source is disposed to induce an thermoacoustic response in said microfluidic channel when holding said sample under test, wherein said ultrasonic transducer is disposed to receive said thermoacoustic response, wherein said ultrasonic transducer outputs a voltage to said appropriately programmed computer, wherein said appropriately programmed computer outputs an analyte value according to said thermoacoustic response.

2) The analyte monitoring device of claim 1, wherein said microfluidic channel comprises materials selected from the group consisting of cross-linked polystyrene, polystyrene (HIPS), polyethylene (LDPE or HDPE), polypropylene, Polytetrafluoroethylene (PTFE), Polyethylene Terephthalate (PET), glass, silicon dioxide, silicon nitride, aluminum oxide, and aluminum nitride.

3) The analyte monitoring device of claim 1, wherein said microfluidic channel comprises a living blood vessel or a living plant vessel.

4) The analyte monitoring device of claim 1, wherein said electromagnetic excitation comprises an applicator comprising material selected from the group consisting of duraluminum, tin, aluminum, titanium, copper, gold, and platinum.

5) The analyte monitoring device of claim 4, wherein said electromagnetic excitation applicator comprises an insulating dielectric covering, wherein said insulating dielectric covering comprises a material selected from the group consisting of glass, silicon oxide, silicon nitride, aluminum oxide, aluminum and nitride.

6) The analyte monitoring device of claim 1 further comprises a housing, wherein said housing comprises insulating dielectric materials selected from consisting of cross-linked polystyrene, polystyrene (HIPS), polyethylene (LDPE or HDPE), polypropylene, Polytetrafluoroethylene (PTFE), and Polyethylene Terephthalate (PET)).

7) The analyte monitoring device of claim 1, wherein said electromagnetic excitation source is selected from the group consisting of an RF source, and an optical source.

8) The analyte monitoring device of claim 1, wherein a magnetic source is disposed proximal to said sample under test, wherein said electromagnetic excitation source is disposed to induce a magnetoacoustic response in said microfluidic channel when holding said sample under test, wherein said ultrasonic transducer is disposed to receive said magnetoacoustic response, wherein said ultrasonic transducer outputs a voltage proportional to said magnetoacoustic response.

9) The analyte monitoring device of claim 8, wherein said appropriately programmed computer is configured to match a waveform shape and amplitude of said thermoacoustic or said magnetoacoustic response in a time domain to known signatures from analytes of interest.

10) The analyte monitoring device of claim 8, wherein said appropriately programmed computer is configured to match a frequency shape and phase content of said thermoacoustic or said magnetoacoustic response in a frequency domain to known signatures from analytes of interest.

11) The analyte monitoring device of claim 8, wherein said sample under test comprises a contrast agent, wherein said contrast agent interacts with an analyte that is disposed to provide an identifiable thermoacoustic response or a magnetoacoustic response, wherein said analyte comprises a biomolecule, cells, or synthetic compounds.

12) The fluid monitoring device of claim 11, wherein said contrast agent comprises materials selected from the group consisting of iron oxide, super-paramagnetic iron oxide nanoparticles (SPION), wherein said SPION is selected from the group consisting of hematite (α—Fe₂O₃), maghaemite (γ—Fe₂O₃), and aluminum substitutes in Iron Oxide (ε—AlₓFe₂—xO₃).

13) The analyte monitoring device of claim 1, wherein said electromagnetic excitation source is modulated, wherein said modulated electromagnetic excitation source is disposed to generate a modulated thermoacoustic response.

14) The analyte monitoring device of claim 1, wherein said ultrasonic transducer comprises a MEMS sensor.

15) The analyte monitoring device of claim 1, wherein said sample under test is selected from the group consisting of blood, saliva, urine, bodily fluids, agricultural fluid extracts, water system, sewer, and industrial fluid extracts.

16) The analyte monitoring device of claim 1, wherein said thermoacoustic response comprises an acoustic shock wave in said microfluidic channel formed by microthermal heating and expansion.
